(19) United States
(12) Patent Application Publication
(10) Pub. No.: US 2004/0247574 A1
(43) Pub. Date: Dec. 9, 2004

(54) Methods for enhancing stem cell engraftment during transplantation

(76) Inventors: Kent W. Christopherson II, Missouri City, TX (US); Giao Hangoc, Brownsburg, IN (US); Hal E. Broxmeyer, Indianapolis, IN (US)

Correspondence Address: DANN, DORFMAN, HERRELL & SKILLMAN, 1601 MARKET STREET, SUITE 2400, PHILADELPHIA, PA, 19103-2307, US

(21) Appl. No.: 10/854,723

(22) Filed: May 26, 2004

Related U.S. Application Data
(60) Provisional application No. 60/473,589, filed on May 27, 2003.

(51) Int. Cl. A61K 48/00

(52) U.S. Cl. 424/93.7

(57) ABSTRACT

The present invention relates to the fields of hematopoietic stem cell transplantation and molecular biology. More specifically, methods for improving engraftment efficiency in stem cell transplants by improving stem cell homing to bone marrow are provided.

### CROSS REFERENCE TO RELATED APPLICATION

This application claims priority to U.S. provisional Application, 60/473,589 filed May 27, 2003, the entire contents of which are incorporated by reference herein.

Pursuant to 35 U.S.C. §202(c), it is acknowledged that the U.S. Government has certain rights in the invention described., which was made in part with funds from the National Institutes of Health, grant number R01DK53674.

### FIELD OF THE INVENTION

The present invention relates to the fields of hematopoietic stem cell transplantation and molecular biology. More specifically, methods for improving engraftment efficiency in stem cell transplants by increasing stem cell homing to bone marrow are provided.

### BACKGROUND OF THE INVENTION

Several publications and patent documents are cited throughout the specification in order to describe the state of the art to which this invention pertains. Each of these references is incorporated herein as though set forth in full.

Stem cell transplants are a critical component of treatment of a wide array of disorders. There is great promise for a wide array of therapeutic benefits from stem cell transplantation. However stem cell transplants can be difficult, costly, and are often very dangerous to the recipient. Stem cells are difficult to harvest and maintain, the donor and recipient need to be close genetic matches (ideally identical twins.) Often efficiency of engraftment in the recipient is low. Accordingly, there is great interest in pursuing methods which will improve the ease and efficacy of stem cell transplantation. The discovery that cytokines and chemokines play a crucial role in Hematopoietic Stem Cell (HSC) mobilization and homing/engraftment provides a means to influence these processes.

CXCL12 (also known as stromal cell-derived factor 1α/SDF-1α) chemoattracts hematopoietic stem and progenitor cells (HSC/HPC) (Aiuti A, et al. J Exp Med. 1997;185:111-120; Kim C H, et al. Blood. 1998;91:100-110; Broxmeyer H E. Int J Hematol. 2001;74:9-17,) and is one of a unique few in the chemokine subfamily of cytokines that binds only one receptor (Nagasawa T, et al. Nature. 1996;382:635-638; Zou Y R, et al. Nature. 1998;393:595-599; Ma Q, et al. Proc Natl Acad Sci USA. 1998;95:9448-9453; Tachibana K, et al. Nature. 1998;393:591-594.) In contrast, redundancy exists in the majority of chemokine/receptor interactions; many receptors are bound by multiple chemokines and many chemokines bind more than one receptor. CXCL12^{-/-} and CXCR4^{-/-} mice share the same phenotype supporting the one chemokine/one receptor hypothesis for CXCL12/CXCR4 (Nagasawa T, et al. Nature. 1996;382:635-638; Zou Y R, et al. Nature. 1998;393:595-599.) CXCL12 is an important component of the mobilization of HSC/HPC from the bone marrow (Kim C H, et al. Blood. 1998;91:100-110; Broxmeyer H E. Int J Hematol. 2001;74:9-17.) However, whether or not CXCL12 is mechanistically involved in G-CSF induced mobilization of HSC/HPC has yet to be determined.

CD26 (DPPIV/dipeptidylpeptidase IV) is a membrane bound extracellular peptidase that cleaves dipeptides from the N-terminus of polypeptide chains after a proline or an alanine (Bongers J, et al. Biochim Biophys Acta. 1992;1122:147-153.) The N-terminus of chemokines is known to interact with the extracellular portion of chemokine receptors. Consequently, the removal of the N-terminal amino acids result in significant changes in receptor binding and/or functional activity (Baggiolini M. Nature. 1998;392:565-568.) There are, however, naturally occurring N-terminal truncated forms of chemokines which have been isolated with full length forms (Pal R, et al. Science. 1997;278:695-698; Struyf S, et al. J Immunol. 1998;161:2672-2675; Struyf S, et al. Eur J Immunol. 1998;28:1262-1271; Wuyts A, et al. Eur J Biochem. 1999;260:421-429; Tensen C P, et al. J Invest Dermatol. 1999;112:716-722; Noso N, et al. Eur J Biochem. 1998;253:114-122; Vulcano M, et al. Eur J Immunol. 2001;31:812-822.)

CD26 cleaves chemokines containing the essential N-terminal X-Pro or X-Ala motif (Oravecz T, et al. J Exp Med. 1997;186:1865-1872; Schols D, et al. Antiviral Res. 1998;39:175-187; Proost P, et al. J Biol Chem. 1998;273:7222-7227; Proost P, et al. FEBS Lett. 1998;432:73-76; Shioda T, et al. Proc Natl Acad Sci USA. 1998;95:6331-6336; Van Coillie E, et al. Biochemistry. 1998;37:12672-12680; Struyf S, et al. J Immunol. 1999;162:4903-4909; Proost P, et al. J Biol Chem. 1999;274:3988-3993; Iwata S, et al. Int Immunol. 1999;11:417-426; Proost P, et al. Blood. 2000;96:1674-1680.) CXCL12, along with CCL22, has been shown to be selectively truncated in vitro by CD26 as compared to other chemokines containing the appropriate X-Pro or X-Ala motif (Lambeir A M, et al. J Biol Chem. 2001;276:29839-29845.) In addition to chemokines, the pancreatic polypeptide family (including neuropeptide Y and peptide YY) and the glucagon family (glucagons, glucagon-like peptide-1, and glucagon-like peptide-2) have also been identified as natural substrates (Mentlein R. Regul Pept. 1999;85:9-24).

CD26 is expressed on many hematopoietic cell populations, including stimulated B and NK cells and activated T-lymphocytes, as well as fibroblasts, and epithelial cells (Vanham G, et al. J Acquir Immune Defic Syndr. 1993;6:749-757; Kahne T, et al. Int J Mol Med. 1999;4:3-15; Huhn J, et al. Immunol Lett. 2000;72:127-132). In addition, CD26 is present in a catalytically active soluble form in plasma (Durinx C, et al. Eur J Biochem. 2000;267:5608-5613). However, very little is known about CD26 expression on normal bone marrow derived HSC/HPC. Since it had been previously established that cord blood is a functional source of transplantable HSC/HPC, (Broxmeyer H E, et al. Proc Natl Acad Sci USA. 989;86:3828-3832;⁻ Gluckman E, et al. N Engl J Med. 1989;321:1174-1178; Broxmeyer H, Smith F. Cord Blood Stem Cell Transplantation. In: Thomas E D, ed. Hematopoietic cell transplantation (ed 2nd). Oxford; Malden, Mass., USA: Blackwell Science; 1999:431-443) CD26 expression patterns were studied in CD34⁺ cells isolated from cord blood (Christopherson K W, 2nd, et al. J Immunol. 2002;169:7000-7008.) Evidence was presented that CD26 is expressed by a subpopulation of normal CD34+ hematopoietic cells isolated from cord blood and that these cells posses CD26 peptidase activity (Christopherson K W, 2nd, et al. J Immunol. 2002;169:7000-7008). More importantly, the functional in vitro studies performed suggested that the process of CXCL12 cleavage by CD26 on a subpopulation of CD34+ cells may represent a novel regulatory mechanism for the entire HSC/HPC population with respect to the migration, homing/engraftment, and mobilization of these cells.

Wallner et al., U.S. Pat. Nos. 6,258,597; 6,300,314; and 6,355,614 are drawn to methods of improving stem cell mobilization by administering Va-boro-pro, which is described as a CD26 inhibitor. However, further studies by this group indicate that the inhibitor administered in these studies, Val-boro-pro is not a specific inhibitor of CD-26, and may be mediating it's effects through other targets and activities.

Given the many applications of hematopoietic stem cell therapy, a need exists in the art for more efficient means of stem cell transplantation.

### SUMMARY OF THE INVENTION

In accordance with the present invention, methods of improving stem cell transplant efficiency by administering a CD26 inhibitor which improves homing, and engraftment are provided. Specifically, a method of improving stem cell engraftment by administering a CD26 inhibitor is disclosed. The CD26 inhibitor of the invention may be administered to hematopoietic stem cells in vitro. The inhibitor of the instant invention may be administered for a short time, for example for 15 minutes to 6 hours. In certain embodiments, the inhibitor is administered for a time period less than that required for cell division. The inhibitor of the invention may be any CD26 inhibitor, including but not limited to Diprotin A (Ile-Pro-Ile), Valine-Pyrrolidide, or any other molecule which inhibits or antagonizes CD26 activity. The inhibitor is preferably administered at a concentration of no less than about 5 mM. The cells are treated at a concentration of 1×10⁶ donor cells per mL.

In another embodiment of the invention, the above method is practiced in combination with administration of a CD26 inhibitor to the stem cell recipient, in vivo.

In yet another embodiment of the invention, the stem cells are obtained from a source comprising a limited number of cells (e.g. cord blood.)

In yet another embodiment, the instant method may be used in autologous or non-autologous HSC transplantation.

In a further embodiment, the method of the instant invention may be administered to myeloablated or non-myeloablated patients. The inhibitor is preferably administered at a concentration of no less than about 1 μMol/kg total body weight. The inhibitor may be administered at a dose of about 1-100 μMol/kg total body weight, or about 1-50 μMol/kg total body weight, or about 1-30 μMol/kg total body weight, or about 1-10 μMol/kg total body weight.

Another aspect of the invention comprises isolated stem cells which have been exposed to a CD26 inhibitor for a time sufficient to inhibit CD26 activity.

### BRIEF DESCRIPTIONS OF THE DRAWINGS

FIG. 1 shows expression of Sca-1 and c-kit on lineage depleted mouse bone marrow (mBM) cells. Expression of these markers was used to gate and sort Sca-1⁺c-kit⁺lin⁻ cells (upper-right quadrant) and Sca-1⁺c-kit⁻lin⁻ cells (upper-left quadrant) for further expression and functional analysis of this population of cells. Sample dot plot shown is representative of data obtained from 6 independent mBM samples.

FIGS. 2A-C depict expression of CD26 and CXCR4. CD26 cell surface expression was measured by flow cytometry on lin⁻ cells using the following fluorochrome-conjugated monoclonal antibodies simultaneously: CD26-FITC, CXCR4-PE, Sca-1-PECy5.5, and c-kit-APC. (FIG. 2A) Representative isotype control is shown. (FIG. 2B) CD26 is expressed on 73% of Sca-1⁺c-kit⁺lin⁻ cells. Simultaneous examination of CXCR4 expression in these cells reveals that the majority of CD26+ and CD26− cells express CXCR4. Sca-1⁺c-kit⁺lin⁻ cells have one distinct population of cells with respect to CD26 expression of which 73% fall in the CD26+ positive category as compared to the isotype control. (FIG. 2C) CD26 is expressed on 75% of Sca-1⁺c-kit⁻lin⁻ cells and of those the majority are CXCR4+. Unlike Sca-1⁺c-kit⁺lin⁻ cells, Sca-1⁺c-kit⁻lin⁻ cells have two distinct CD26+ and CD26-populations. Data obtained from 6 independent mBM samples indicate that a significant percentage of normal HSC/HPC from mBM express CD26. Representative sample Sca-1⁺c-kit⁺lin⁻ (B) and Sca-1⁺c-kit⁻lin⁻ (C) dot plots are shown.

FIGS. 3A-B show CD26 peptidase activity. The CD26 peptidase activity of sorted Sca-1⁺c-kit⁺lin⁻ (FIG. 3A) and Sca-1⁺c-kit⁻lin⁻ (FIG. 3B) mBM cells was measured. Using the chromogenic substrate Gly-Pro-pnitoanilide (Gly-Pro-pNA) the production of pNA by DPPIV cleavage was monitored. The results are plotted as pmoles pNA produced versus minutes and slope was calculated at the linear portion of the enzymatic curve giving a measure of CD26 peptidase activity expressed as U/1000 cells where 1 U=1 pmole pNA/min. (FIG. 3A) Sca-1⁺c-kit⁺lin⁻ mBM cells have CD26 activity (207.97 U/1000 cells, n=8). (FIG. 3B) Approximately the same peptidase activity was recorded for Sca-1⁺c-kit⁻lin⁻ mBM cells (193.28 U/1000 cells, n=8).

FIGS. 4A-B depict a migratory response to N-terminal truncated CXCL12 (amino acids 3-68), produced by CD26 cleavage. Chemotaxis assays using Sca-1⁺c-kit⁺lin mBM cells (FIG. 4A) and Sca-1⁺c-kit⁺lin⁻ mBM cells (FIG. 4B) were performed comparing the normal CXCL12 and the N-terminal truncated form CXCL12 (3-68). (FIG. 4A) CXCL12 induced a normal dose dependent migratory response in Sca-1⁺c-kit⁺lin⁻ mBM cells (circle). CXCL12(3-68) did not induce the migration of cells compared to CXCL12 (square, p<0.01, n=8). Pre-incubation of cells for 15 minutes with CXCL12(3-68) inhibits the normal CXCL12 induced migration of cells (triangle, p=0.04, n=8). (FIG. 4B) CXCL12 again induced a normal dose dependent migratory response in Sca-1⁺c-kit⁻lin⁻ mBM cells (circle). CXCL12(3-68) did not induce the migration of cells compared to CXCL12 (square, p<0.01, n=8). Pre-incubation of cells for 15 minutes with CXCL12(3-68) inhibits the normal CXCL12 induced migration of cells (triangle, p=0.02, n=8).

FIGS. 5A-B show the effect of CD26 inhibition on CXCL12 induced migration. Chemotaxis assays induced by CXCL12 were performed comparing the control untreated Sca-1⁺c-kit⁺lin⁻ mBM cells (FIG. 5A) with Sca-1⁺c-kit⁺lin⁻ mBM cells exposed to Diprotin A (FIG. 5B). (FIG. 5A) CXCL12 induced a normal dose dependent migratory response in untreated Sca-1⁺c-kit⁺lin⁻ mBM cells (circle). Treatment with 5 mM Diprotin A (Ile-Pro-Ile) was observed to enhance the migratory response of Sca-1⁺c-kit⁺lin⁻ mBM cells to CXCL12 (square n=8,p=0.03). The enhancement with Diprotin A treatment is equivalent to a 1.7-fold increase in total cell migration in response to 200 and 400 ng/ml CXCL12 and two-fold at 100 ng/ml CXCL12. (FIG. 5B) CXCL12 induced a normal dose dependent migratory response in Sca-1⁺c-kit⁻lin⁻ mBM cells (circle). Treatment with Diprotin A also enhanced the migratory response of cells to CXCL12 (n=8, p=0.02). The enhancement with Diprotin A treatment is equivalent to a two-fold increase in total cell migration in response to 200 and 400 ng/ml CXCL12 and 2.5-fold at 100 ng/ml.

FIGS. 6A-C show G-CSF induced mobilization of HSC/HPC in C57BL/6 mice. Data is plotted as a % mobilization, where G-CSF is equal to 100% for each progenitor subtype. Treatment with either Diprotin A alone or Val-Pyr was observed to have little or no effect on the mobilization of progenitors. Dipotin A or Val-Pyr treatment during G-CSF mobilization resulted in a significant reduction in (FIG. 6A) CFU-GM (p<0.01), (FIG. 6B) BFU-E (p=0.06), and (FIG. 6C) CFU-GEMM (p=0.01) compared to G-CSF alone.

FIGS. 7A-C shows G-CSF induced mobilization of HSC/HPC in DBA/2 mice. Data is plotted as a % mobilization, where G-CSF is equal to 100% for each progenitor subtype. Treatment with either Diprotin A alone or Val-Pyr was observed to have little or no effect on the mobilization of progenitors. Dipotin A or Val-Pyr treatment during G-CSF mobilization resulted in a significant reduction in (FIG. 7A) CFU-GM (p<0.01), (FIG. 7B) BFU-E (p=0.02), and (FIG. 7C) CFU-GEMM (p<0.01) compared to G-CSF alone.

FIGS. 8A-C show G-CSF induced mobilization of progenitors in WT C57BL/6 and CD26^{-/-} mice. Mobilization data is plotted as progenitors/ml of peripheral blood. No statistical difference in the number of CFU-GM (FIG. 8A, p=0.51), BFU-E (FIG. 8B, p=0.11), or CFU-GEMM (FIG. 8C, p=0.32) in the periphery was observed between untreated WT mice and untreated CD26^{-/-} mice. Significant mobilization of CFU-GM (A, p<0.01), BFU-E (B, p<0.01), and CFU-GEMM (C, p<0.01) was observed in WT mice in response to G-CSF treatment compared to untreated WT mice. The number of CFU-GM (A, p=0.01), BFU-E (B, p<0.01), and CFU-GEMM (C, p=0.01) in the periphery of CD26^{-/-} mice was slightly, but significantly, greater than untreated CD26^{-/-} mice. Little or no difference in mobilization of CFU-GM (A, p=0.05), BFU-E (B, p=0.32), and CFU-GEMM (C, p=0.36) was observed in CD26^{-/-} mice compared to untreated WT mice.

FIG. 9 shows the enhanced migration observed when utilizing CD26^{-/-} mouse bone marrow derived HSC (as defined as being contained within the Sca-1⁺lin⁻ population). Inhibition or loss of CD26 increases CXCL12 induced chemotaxis of Sca-1⁺lin⁻ mouse BM cells. Diprotin A treated C57BL/6 Sca-1⁺lin⁻ mouse BM cells (-▪-) and CD26^{-/-} cells (-▴-) had a greater CXCL12 induced migratory response than control untreated C57BL/6 cells (-●-) (P<0.01). Diprotin A treatment of CD26^{-/-} cells (-♦-) had no effect compared to untreated CD26^{-/-} cells (-▴-). n=8

FIGS. 10A-C show that CD26 inhibitor treated cells and CD26^{-/-} cells exhibit greater short-term homing to the recipient's bone marrow (BM). Donor contribution to Sca-1+lin− cells in recipient mouse's BM was determined by flow cytometry 24 hours post transplant, utilizing antibodies to CD45.2 (expressed on the donor cell population) and CD45.1 (expressed on the residual recipient cell population). (FIG. 10A) Sorted Sca-1⁺lin⁻ C57Bl/6 cells treated with 5 mM Diprotin A for 15 minutes prior to transplantation and CD26^{-/-} cells have increased short-term homing into BoyJ recipient mice, as compared to control C57BL/6 cells. (P<0.05) n=5 (FIG. 10B) Sca-1⁺lin⁻ cells within the donor low density BM (LDBM) unit treated with either CD26 inhibitor (Diprotin A or Val-Pyr) prior to transplant or the transplantation of CD26^{-/-} cells, resulted in a significant increase in short-term homing of donor cells, into the BoyJ recipients (P<0.01). n=6 (FIG. 10C) The increase in homing efficiency of Sca-1⁺lin⁻ HSC within donor LDBM cells observed with CD26 inhibitor (Diprotin A) treatment of C57BL/6 or with CD26^{-/-} donor cells is reversible by treatment with a CXCR4 antagonist, AMD3100, for 15 minutes prior to transplant. AMD3100 also reduces the homing efficiency of C57BL/6 donor cells in the absence of any CD26 inhibitor. (P<0.05) n=5

FIG. 11 describes the loss of CD26 activity upon CD26 inhibitor treatment. CD26 peptidase activity (U/1000 cells where 1 U=1 pmol pNA/minute) of C57BL/6 BM cells is rapidly lost with inhibitor treatment (P<0.01). After cells were washed 15 minutes post inhibitor treatment cells begin to recover within 4 hours.

FIGS. 12A-C demonstrate that CD26^{-/-} donor cells have enhanced long-term engraftment as compared to control C57BL/6 donor cells at limiting cell dilutions and result in increased recipient mouse survival (FIG. 12A). As measured by percent donor contribution to the formation of peripheral blood leukocytes, transplantation of CD26^{-/-} donor cells into congenic BoyJ recipient mice resulted in a significant increase in non-competitive long-term engraftment as compared to the transplantation of control C57BL/6 donor cells at six months post transplant (P<0.01). n=3-5 (FIGS. 12B & 12C) Corresponding mouse survival curves following transplantation of limiting dilutions of cells. Increased overall survival is observed 60 days post transplant in those recipient mice receiving low numbers of transplanted CD26^{-/-} cells (C) versus control C57BL/6 cells (B) (P<0.01). n=3-5

FIGS. 13A-B show that CD26 inhibitor treated cells exhibit greater long-term engraftment. Donor contribution to functional hematopoiesis in the recipient's bone marrow (BM) was determined by flow cytometric analysis of peripheral blood (PB) cells 6 months post transplant, utilizing antibodies to CD45.2 (expressed on the donor cell population) and CD45.1 (expressed on the residual recipient cell population). (FIG. 13A) Increased donor cell contribution to the formation of peripheral blood leukocytes during non-competitive long term engraftment assays was also observed with CD26 inhibitor treatment (Diprotin A or Val-Pyr) at six months post transplant (P<0.05) (n=5). (FIG. 13B) An even greater increase in donor contribution to chimerism was observed in secondary transplanted mice receiving cells treated with CD26 inhibitors prior to primary transplant, relative to untreated control cells six months post transplant (P<0.01) n=5

FIGS. 14A-B show that CD26 inhibitor treated donor cells and CD26^{-/-} donor cells have increased competitive repopulation and increased engraftment of secondary repopulating HSC as compared to control donor cells. (FIG. 14A) Increased donor cell contribution to chimerism in competitive repopulation assays was observed with CD26 inhibitor treatment (Diprotin A or Val-Pyr) of either 5×10⁵ or 2.5×10⁵ donor cells by monitoring the percent donor contribution of C57BL/6 donor cells to the formation of peripheral blood leukocytes in direct competition with a constant number (5×10⁵) of recipient matched competitor BoyJ cells (P<0.01). CD26^{-/-} donor cells had a greatly enhanced donor contribution to chimerism at all donor cell numbers. (P<0.01). n=5 (FIG. 14B) An even greater increase in donor cell contribution was observed with CD26 inhibitor treated donors and CD26^{-/-} donors in secondary transplanted recipient BoyJ mice (P<0.01). n=5

### DETAILED DESCRIPTION OF THE INVENTION

The invention disclosed herein is important to all diseases where standard treatment involves stem cell transplantation, such as but not limited to AML, ALL, CLL, and CML, as well as diseases in which transplantation is considered after relapse, such as Hodgkin's Lymphoma and NH Lymphoma (Bolwell, B J Current Controversies in Bone Marrow Transplantation. In: Current Clinical Oncology. Totowa, N.J.: Humana Press; 2000). Increasing the efficiency of transplantation regardless of the donor source is important, and becomes extremely important in the case of cord blood transplantation. Thus far, cord blood use in transplantation has been primarily confined to children, due to the small sample size. To counter this problem, many laboratories have evaluated ex-vivo stem cell expansion procedures, but the initial results are discouraging (Broxmeyer, H et al. Cord Blood Stem Cell Transplantation. In: Hematopoietic cell transplantation (ed 2^{nd}) Oxford; Malden, Mass., USA:Blackwell Science; 1999:431-443). Since not all stem cells home to the appropriate marrow niches necessary for engraftment, a need exists in the art for improvement of stem cell homing efficiency.

Provided herein is evidence of the role of CD26 in stem cell mobilization and homing. Also provided herein are methods of improving stem cell transplant efficiency by administering a CD26 inhibitor.

Specifically, evidence is presented that CD26 is expressed by a subpopulation of Sca-1⁺c-kit⁺lin⁻ cells isolated from mouse bone marrow, as well as Sca-1⁺c-kit⁻lin⁻ cells, and that these cells have CD26 peptidase activity. Mouse marrow cells were chosen because mice represent a useful model for in vivo mobilization studies. In vitro functional studies showed that the N-terminal truncated CXCL12 lacks the ability to induce the migration of both Sca-1⁺c-kit⁺lin⁻ and Sca-1⁺c-kit⁻lin⁻ mouse marrow cells. In addition, it acts as an inhibitor, resulting in the reduction of migratory response to normal full length CXCL12. Inhibiting the endogenous CD26 activity on Sca-1⁺c-kit⁺lin⁻ and Sca-1⁺c-kit⁻lin⁻ mouse marrow cells with a specific CD26 inhibitor enhances the chemotactic response of these cells to CXCL12. In support of similar activity in vivo, treatment of mice with CD26 inhibitors during G-CSF induced mobilization resulted in a reduction in the number of progenitor cells in the peripheral blood. This reduction in the number of progenitor cells mobilized provides evidence that CD26 plays a role in G-CSF mobilization. Finally, evidence is provided demonstrating improved short and long-term stem cell engraftment and competitive repopulation ability of donor cells after treatment with a CD26 inhibitor or by use of CD26^{-/-} cells in accordance with the methods of the present invention.

### Definitions

The following definitions are provided to facilitate an understanding of the present invention:

“CD26”, (DPPIV/dipeptidylpeptidase IV) is a membrane bound extracellular peptidase that cleaves dipeptides from the N-terminus of polypeptide chains after a proline or an alanine. “CD26 activity” or “DPPIV activity” encompasses any activity of CD26, including peptidase activity. “CD26” or “DPPIV” inhibitor or antagonist refers to any substance, chemical, biological, and so forth, which is capable of inhibiting CD26 activity. Preferably, CD26 or DPPIV inhibitors and antagonists inhibit CD26 peptidase activity, at levels sufficient to improve stem cell homing.

“Short term exposure” refers to exposure of stem cells to a CD26 inhibitor for a short period of time, for example, for less time than is required for cell expansion to occur. Alternatively, short term exposure means for a time period of about 5 minutes to about 12 hours. Preferably, “short term exposure” is from 15 minutes to 6 hours.

“Homing” refers to localization to a particular area, for example localization of transplanted stem cells to the bone marrow.

“Donor” refers to the organism donating the therapeutic stem cells.

“Recipient” is the patient receiving the therapeutic stem cells.

“Stem cell” or “hematopoietic stem cell” means a pluripotent cell of the hematopoietic system capable of differentiating into a cell of a specific lineage, such as lymphoid, or myeloid.

“Transfected stem cell” or “transduced stem cell” describes a stem cell into which exogenous DNA or an exogenous DNA gene has been introduced, for example by retroviral infection.

The term “engrafting” or “engraftment” means the persistence of proliferating stem cells in a particular location over time.

The term “myeloablated” refers to a patient who has undergone irradiation, or other treatment, such as chemotherapeutic treatment, to cause the death of at least 50% of the bone marrow cells of the patient.

“Non-myeloablated” refers to a patient who has not undergone irradiation, or other treatment (such as chemotherapy) to cause the death of the bone marrow cells of the mammal.

The term “autologous” describes nuclear genetic identity between donor cells or tissue and those of the recipient.

“Multipotent” means that a cell is capable, through its progeny, of giving rise to several different cell types found in the adult animal.

“Pluripotent” means that a cell is capable, through its progeny, of giving rise to all of the cell types which comprise the adult animal including the germ cells. Both embryonic stem and embryonic germ cells are pluripotent cells under this definition.

The term “transgenic” animal or cell refers to animals or cells whose genome has been subject to technical intervention including the addition, removal, or modification of genetic information. The term “chimeric” also refers to an animal or cell whose genome has modified.

The term “knockout mouse” refers to a mouse with a DNA sequence introduced into it's germline by way of human intervention, preferably a sequence which is designed to specifically alter cognate endogenous alleles. Preferably a targeted gene has been “knocked out” to assess the biological and functional consequences of elimination of such target genes. Such mice also provide an ideal in vivo model for assessing restoration of the lost phenotype via complementation with cognate alleles from the same or different species using recombinant DNA techniques.

The term “totipotent” as used herein may refer to a cell that gives rise to a live born animal. The term “totipotent” may also refer to a cell that gives rise to all of the cells in a particular animal. A totipotent cell may give rise to all of the cells of an animal when it is utilized in a procedure for developing an embryo from one or more nuclear transfer steps. Totipotent cells may also be used to generate incomplete animals such as those useful for organ harvesting, e.g., having genetic modifications to eliminate growth of an organ or appendage by manipulation of a homeotic gene.

The term “cultured” as used herein in reference to cells may refer to one or more cells that are undergoing cell division or not undergoing cell division in an in vitro environment. An in vitro environment may be any medium known in the art that is suitable for maintaining cells in vitro, such as suitable liquid media or agar, for example. Specific examples of suitable in vitro environments for cell cultures are described in the art (Culture of Animal Cells: a manual of basic techniques (3.sup.rd edition), 1994, R. I. Freshney (ed.), Wiley-Liss, Inc.; Cells: a laboratory manual (vol. 1), 1998, D. L. Spector, R. D. Goldman, L. A. Leinwand (eds.), Cold Spring Harbor Laboratory Press; and Animal Cells: culture and media, 1994, D. C. Darling, S. J. Morgan John Wiley and Sons, Ltd).

The term “cell line” as used herein may refer to cultured cells that can be passaged at least one time without terminating. The invention relates to cell lines that can be passaged at least 1, 2, 5, 10, 15, 20, 30, 40, 50, 60, 80, 100, and 200 times. Cell passaging is defined hereafter.

The term “suspension” as used herein may refer to cell culture conditions in which cells are not attached to a solid support. Cells proliferating in suspension may be stirred while proliferating.

The term “monolayer” as used herein may refer to cells that are attached to a solid support while proliferating in suitable culture conditions. A small portion of cells proliferating in a monolayer under suitable growth conditions may be attached to cells in the monolayer but not to the solid support. Preferably less than 15% of these cells are not attached to the solid support, more preferably less than 10% of these cells are not attached to the solid support, and most preferably less than 5% of these cells are not attached to the solid support.

The term “plated” or “plating” as used herein in reference to cells may refer to establishing cell cultures in vitro. For example, cells may be diluted in cell culture media and then added to a cell culture plate, dish, or flask. Cell culture plates are commonly known to a person of ordinary skill in the art. Cells may be plated at a variety of concentrations and/or cell densities.

The term “cell plating” may also extend to the term “cell passaging.” Cells of the invention may be passaged using cell culture techniques well known to those skilled in the art. The term “cell passaging” may refer to a technique that involves the steps of (1) releasing cells from a solid support or substrate and disassociation of these cells, and (2) diluting the cells in media suitable for further cell proliferation. Cell passaging may also refer to removing a portion of liquid medium containing cultured cells and adding liquid medium to the original culture vessel to dilute the cells and allow further cell proliferation. In addition, cells may also be added to a new culture vessel which has been supplemented with medium suitable for further cell proliferation.

The term “proliferation” as used herein in reference to cells may refer to a group of cells that can increase in number over a period of time.

The term “isolated” as used herein may refer to a cell that is mechanically separated from another group of cells. Examples of a group of cells are a developing cell mass, a cell culture, a cell line, and an animal.

The term “differentiated cell” as used herein may refer to a precursor cell that has developed from an unspecialized phenotype to a specialized phenotype.

The term “undifferentiated cell” as used herein may refer to a precursor cell that has an unspecialized phenotype and is capable of differentiating. An example of an undifferentiated cell is a stem cell.

The term “asynchronous population” as used herein may refer to cells that are not arrested at any one stage of the cell cycle. Many cells can progress through the cell cycle and do not arrest at any one stage, while some cells can become arrested at one stage of the cell cycle for a period of time. Some known stages of the cell cycle are G1, S, G2, and M. An asynchronous population of cells is not manipulated to synchronize into any one or predominantly into any one of these phases. Cells can be arrested in the M stage of the cell cycle, for example, by utilizing multiple techniques known in the art, such as by colcemid exposure. Examples of methods for arresting cells in one stage of a cell cycle are discussed in WO 97/07669, entitled “Quiescent Cell Populations for Nuclear Transfer”.

The term “modified nuclear DNA” as used herein may refer to a nuclear deoxyribonucleic acid sequence of a cell, embryo, fetus, or animal of the invention that has been manipulated by one or more recombinant DNA techniques. Examples of recombinant DNA techniques are well known to a person of ordinary skill in the art, which may include (1) inserting a DNA sequence from another organism (e.g., a human organism) into target nuclear DNA, (2) deleting one or more DNA sequences from target nuclear DNA, and (3) introducing one or more base mutations (e.g., site-directed mutations) into target nuclear DNA. Cells with modified nuclear DNA may be referred to as “transgenic cells” or “chimeric cells” for the purposes of the invention. Transgenic cells can be useful as materials for nuclear transfer cloning techniques provided herein. The phrase “modified nuclear DNA” may also encompass “corrective nucleic acid sequence(s)” which replace a mutated nucleic acid molecule with a nucleic acid encoding a biologically active, phenotypically normal polypeptide. The constructs utilized to generate modified nuclear DNA may optionally comprise a reporter gene encoding a detectable product.

As used herein, the terms “reporter,” “reporter system”, “reporter gene,” or “reporter gene product” shall mean an operative genetic system in which a nucleic acid comprises a gene that encodes a product that when expressed produces a reporter signal that is a readily measurable, e.g., by biological assay, immunoassay, radioimmunoassay, or by calorimetric, fluorogenic, chemiluminescent or other methods. The nucleic acid may be either RNA or DNA, linear or circular, single or double stranded, antisense or sense polarity, and is operatively linked to the necessary control elements for the expression of the reporter gene product. The required control elements will vary according to the nature of the reporter system and whether the reporter gene is in the form of DNA or RNA, but may include, but not be limited to, such elements as promoters, enhancers, translational control sequences, poly A addition signals, transcriptional termination signals and the like.

“Selectable marker” as used herein refers to a molecule that when expressed in cells renders those cells resistant to a selection agent. Nucleic acids encoding selectable marker may also comprise such elements as promoters, enhancers, translational control sequences, poly A addition signals, transcriptional termination signals and the like. Suitable selection agents include antibiotics such as kanamycin, neomycin, and hygromycin.

Methods and tools for insertion, deletion, and mutation of nuclear DNA of mammalian cells are well-known to a person of ordinary skill in the art. See, Molecular Cloning, a Laboratory Manual, 2nd Ed., 1989, Sambrook, Fritsch, and Maniatis, Cold Spring Harbor Laboratory Press; U.S. Pat. No. 5,633,067, “Method of Producing a Transgenic Bovine or Transgenic Bovine Embryo,” DeBoer et al., issued May 27, 1997; U.S. Pat. No. 5,612,205, “Homologous Recombination in Mammalian Cells,” Kay et al., issued Mar. 18, 1997; and PCT publication WO 93/22432, “Method for Identifying Transgenic Pre-Implantation Embryos”; WO 98/16630, Piedrahita & Bazer, published Apr. 23, 1998, “Methods for the Generation of Primordial Germ Cells and Transgenic Animal Species. These methods include techniques for transfecting cells with foreign DNA fragments and the proper design of the foreign DNA fragments such that they effect insertion, deletion, and/or mutation of the target DNA genome.

Any of the cell types defined herein may be altered to harbor modified nuclear DNA.

Examples of methods for modifying a target DNA genome by insertion, deletion, and/or mutation are retroviral insertion, artificial chromosome techniques, gene insertion, random insertion with tissue specific promoters, homologous recombination, gene targeting, transposable elements, and/or any other method for introducing foreign DNA. Other modification techniques well known to a person of ordinary skill in the art include deleting DNA sequences from a genome, and/or altering nuclear DNA sequences. Examples of techniques for altering nuclear DNA sequences are site-directed mutagenesis and polymerase chain reaction procedures. Therefore, the invention relates in part to mammalian cells that are simultaneously totipotent and transgenic.

The term “recombinant product” as used herein may refer to the product produced from a DNA sequence that comprises at least a portion of the modified nuclear DNA. This product may be a peptide, a polypeptide, a protein, an enzyme, an antibody, an antibody fragment, a polypeptide that binds to a regulatory element (a term described hereafter), a structural protein, an RNA molecule, and/or a ribozyme, for example. These products are well defined in the art.

The term “promoters” or “promoter” as used herein may refer to a DNA sequence that is located adjacent to a DNA sequence that encodes a recombinant product. A promoter is preferably linked operatively to an adjacent DNA sequence. A promoter typically increases an amount of recombinant product expressed from a DNA sequence as compared to an amount of the expressed recombinant product when no promoter exists. A promoter from one organism may be utilized to enhance recombinant product expression from a DNA sequence that originates from another organism. In addition, one promoter element may increase an amount of recombinant products expressed for multiple DNA sequences attached in tandem. Hence, one promoter element may enhance the expression of one or more recombinant products. Multiple promoter elements are well-known to persons of ordinary skill in the art.

The term “enhancers” or “enhancer” as used herein may refer to a DNA sequence that is located adjacent to the DNA sequence that encodes a recombinant product. Enhancer elements are typically located upstream of a promoter element or can be located downstream of a coding DNA sequence (e.g., a DNA sequence transcribed or translated into a recombinant product or products). Hence, an enhancer element can be located 100 base pairs, 200 base pairs, or 300 or more base pairs upstream or downstream of a DNA sequence that encodes recombinant product. Enhancer elements may increase an amount of recombinant product expressed from a DNA sequence above increased expression afforded by a promoter element. Multiple enhancer elements are readily available to persons of ordinary skill in the art.

The terms “transfected” and “transfection” as used herein refer to methods of delivering exogenous DNA into a cell. These methods involve a variety of techniques, such as treating cells with high concentrations of salt, an electric field, liposomes, polycationic micelles, or detergent, to render a host cell outer membrane or wall permeable to nucleic acid molecules of interest. These specified methods are not limiting and the invention relates to any transformation technique well known to a person of ordinary skill in the art.

The term “antibiotic” as used herein may refer to any molecule that decreases growth rates of a bacterium, yeast, fungi, mold, or other contaminants in a cell culture. Antibiotics are optional components of cell culture media. Examples of antibiotics are well known in the art. See, Sigma and DIFCO catalogs.

### Methods of Improving the Efficiency of Stem Cell Engraftment

Provided herein are methods of improving stem cell engraftment by administering a CD26 inhibitor.

Stem cells may be obtained by various techniques. For example cells may be from an autologous donor (the patient who will receive the cells, or their identical twin), or from a non-autologous donor. Stem cells may be harvested from the bone marrow, obtained from cord blood, or isolated from peripheral blood cells (following G-CSF mobilizing agent treatment.)

These cells are then exposed to a CD26 inhibitor in vitro. Exemplary CD26 inhibitors may include, but are not limited to Diprotin A (Ile-Pro-Ile), Valine-Pyrrolidide, or any other molecule which inhibits or antagonizes CD26 activity. The inhibitor of the instant invention may be administered for a short time (e.g. from about 15 minutes to about 6 hours.) Alternatively the inhibitor is administered for a time sufficient to inhibit CD26 activity, but insufficient for cell expansion to occur.

The inhibitor is preferably administered in a concentration of no less than about 5 mM. The cells are treated at a concentration of 1×10⁶ donor cells per mL.

The CD26 inhibitor treated stem cells are administered to the recipient in need thereof.

Optionally, the patient (recipient) is administered a CD26 inhibitor in vivo prior to, or during transplant. The CD26 inhibitor may be administered at a dose of about 1-10 μmol/kg total body weight. The inhibitor is preferably administered at a concentration of no less than about 1 μMol/kg total body weight. The inhibitor may also be administered at a dose of about 1-100 Mol/kg total body weight, or about 1-50 μMol/kg total body weight, or about 1-30 μMol/kg total body weight.

### Kits for Methods of Enhancing Stem Cell Engraftment

The practice of the invention can be facilitated via incorporation of suitable reagents for enhancing HSC engraftment in a kit. The kits may contain any or all of a CD26 inhibitor, an antibody which binds a specific type of stem cell, a vessel for incubation of stem cells, means for administering stem cells to a patient, or any combination thereof.

The kits may optionally comprise any or all of a polynucleotide, an oligonucleotide, a polypeptide, a peptide, an antibody, a label, marker, or reporter, a pharmaceutically acceptable carrier, a physiologically acceptable carrier, instructions for use, a container, a vessel for administration, an assay substrate, or any combination thereof.

The following non-limiting examples are provided to further illustrate the present invention.

### EXAMPLE 1

### CD26 Activity Modulates Stem Cell Engraftment

### Materials and Methods

Preparation of Mouse Cells

Mouse bone marrow (mBM) cells are flushed from femurs of 6-8 week old mice. Peripheral blood stem cells (PBSC) were collected from 6-8 week old mice by heart stick using 25G needle containing 100 μl Heparin (1000 U/ml). Mononuclear cells (MNC) were isolated by density centrifugation using Lympholyte M (Cedarlane Laboratories, Ontario). Lin⁺ cells (cocktail of monoclonal antibodies for Ly-1, CD45R/B220, CD11b/Mac-1, TER119, Gr-1, 7-4) were depleted using a density particle murine progenitor enrichment cocktail (Stem Cell Technologies, Vancouver, BC). Four-color flow cytometry was then performed, as described below, and Sca-1⁺c-kit⁺lin⁻ and Sca-1⁺c-kit⁻lin⁻ cells were simultaneously sorted. Sorted cell populations, typically 98.3±0.62% pure compared to isotype controls, were then used immediately. Normal C57BL/6 mice were purchased from Harlan (Indianapolis, Ind.). DBA/2 mice were obtained from Jackson Labs (Bar Harbor, Me.). CD26^{-/-} mice (on a C57BL/6 backgound) were obtained from Dr. N. Wagtmann (Novo Dordisk, Denmark) with approval from Dr. D. Marguet (Centre d'Immunologie de Marseille Luminy-INSERM, France) (Marguet D, et al, PNAS USA. 2000;97:6874-6879; Wang M, et al, J Gastroenterol Hepatol. 2002;17:66-71).

CD26 and CXCR4 Expression

CD26 cell surface expression was measured by four-color flow cytometry. Isolated lin− mouse MNCs were stained with mouse CD26 FITC, CXCR4 PE, Sca-1 PE-Cy5.5, and c-kit APC (from either BD Biosciences, San Diego, Calif. or Caltag Laboratories, Burlingame, Calif.). Cells were labeled as described previously and then one hundred thousand events were accumulated for each analysis (Christopherson K W, 2nd, et al. J Immunol. 2002;169:7000-7008; Christopherson K W, 2nd, et al. Blood. 2001;98:3562-3568). The staining protocol was as follows. Cells were first washed in PBS/Pen/Strep/1% BSA and resuspended in 100 μl PBS/Pen/Strep/1% BSA containing the appropriate antibodies. Samples were mixed, and incubated at 4° C. in the dark for 40 minutes. The cells were then washed twice in PBS/Pen/Strep/1% BSA and fixed in PBS/1% paraformaldehyde for subsequent flow cytometric analysis. Six mBM samples were analyzed separately.

CD26/DPPIV Peptidase Activity

CD26 peptidase activity of sorted cells was measured in 96 well microplates using the chromogenic substrate Gly-Pro-p-nitoanilide (Gly-Pro-pNA) (Sigma, St Louis, Mo.) as previously reported (Christopherson K W, 2nd, et al. J Immunol. 2002;169:7000-7008; Kojima K, et al. J Chromatogr. 1980;189:233-240; Nagatsu T, et al. Anal Biochem. 1976;74:466-476). Peptidase activity is expressed as pmoles/min (U) per 1000 cells. Proteolytic activity was determined by measurement of the amount of p-nitroanilide (pNA) formed in the supernatant at 405 nm. One thousand cells per well in the 96-well flat-bottomed plate were incubated at 37° C. with 4 mM Gly-Pro-pNA in 100 μL PBS buffer (pH 7.4) containing 10 mg/ml BSA. Absorbance was measured at 405 nm on a microplate spectrofluorometer (SpectraMax 190, Molecular Devices, Sunnyvale, Calif.) every two minutes and pmoles of pNA formed were calculated by comparison to a pNA standard curve. The results were plotted as pmoles pNA versus minutes and the slope was calculated at the linear portion of the curve giving a measure of DPPIV activity expressed as pmoles/min (U) per 1000 cells. Tests were run using three separate samples (n=3 for each sample); cell-free blanks, substrate-free blanks were run in parallel.

Migration of Hematopoietic Stem Cells

Chemotaxis assays were performed using 96-well chemotaxis chambers (NeuroProbe, Gaithersburg, Md.) in accordance with manufacturer's instructions as described previously with minor variations (Christopherson K W, 2nd, et al. J Immunol. 2002;169:7000-7008; Christopherson K W, 2nd, et al. Blood. 2001;98:3562-3568; Christopherson K, 2nd, et al. Immunol Lett. 1999;69:269-273). Briefly 300 μl of RPMI supplemented with 10% FBS and either 0, 100, 200, and 400 ng/ml of mouse CXCL12 chemokine was added to the lower chamber. Ten-thousand sorted cells in 50 μL of media were added to the upper side of the membrane (5.7 mm diameter, 5 μm pore size, polycarbonate membrane.)

Total cell number in the lower well was obtained by counting using hemocytometer after 4 hours of incubation at 37° C., 5% CO₂. Percent migration was calculated by dividing the number of the cells in the lower well by the total cell input multiplied by 100 and subtracting random migration (always less than 5%) to the lower chamber without chemokine presence. Three samples were analyzed separately in triplicate, and then the data averaged for statistical analysis.

The N-terminal truncated mouse CXCL12 (CXCL12(amino acids 3-68)) was produced by treatment of mouse CXCL12 with DPPIV (Enzyme Systems Products, Livermore, Calif.) for 18 hrs at 37° C. in PBS pH 7.4. Efficiency of the DPPIV digestion of human CXCL12 under these conditions was previously determined to be 100% by mass spectroscopy (Christopherson K W, 2nd, et al. J Immunol. 2002; 169:7000-7008). Chemotaxis assay wells containing truncated CXCL12(3-68) alone were performed using a full dose response of either 0,100, 200, or 400 ng/ml, Chemotaxis assays examining the inhibitory effect of truncated CXCL12(3-68) used either 0, 100, 200, and 400 ng/ml of CXCL12 and a pre-treatment of 100 ng/ml of CXCL12(3-68) for 15 minutes. A 15 minute pre-treatment represents the minimum setup time for the chemotaxis assay after addition of CXCL12(3-68) to the system. Inhibition of endogenous CD26/DPPIV activity was accomplished by pretreatment of cells with 5 mM Diprotin A (Peptides International, Louisville, Ky.) for 15 minutes at 37° C. Diprotin A was allowed to remain in the chemotaxis chamber during the assay. Chemotaxis assays were performed with and without Diprotin A in conjunction with a CXCL12 dose response between 0 and 400 ng/ml.

Mobilization

Mobilization was achieved by treating mice with 2.5 μg (micrograms) G-CSF/mouse 2×/day s.c. for 2 days (Broxmeyer H E, et al. J Exp Med. 1999;189:1987-1992). Mice simultaneously treated with a specific CD26 inhibitor also underwent treatment with either 5 μmol Diprotin A/mouse 2×/day s.c. for 2 days or 5 μmol Val-Pyr/mouse 2×/day s.c. for 2 days. Diprotin A was obtained commercially (Peptides International, Louisville, Ky.) and Val-Pyr was obtained as a gift from Nicolai Wagtmann (Novo Nordisk, Denmark). After either G-CSF or G-CSF plus CD26 inhibitor treatment peripheral blood cells were collected and total cellular nuclearity was measured and progenitor cell assays were performed using total peripheral blood cells.

Progenitor Cells Assays

One-hundred thousand mouse G-CSF mobilized peripheral blood cells were plated in triplicate for colony formation by CFU-GM, BFU-E, and CFU-GEMM and scored at 7 days incubation as previously described (Cooper S, Broxmeyer H E. Measurement of interleukin-3 and other hematopoietic growth factors, such as GM-CSF, G-CSF, M-CSF, erkythropoietin and the potent co-stimulating cyktokines steel factor and FLt-3 ligand. In: Coligan J E K A, Margulies D H, Shevach E M, Strober E, Coico R, ed. Current protocols in immunology. New York: John Wiley & Sons; 1996:6.4.1-6.4.12). Cells were plated for colony formation in 1% methylcellulose culture medium containing 30% FBS, 1 U/ml recombinant(r) human Epo, 0.1 mM Hemin, 5% Pokeweed Mitogen Spleen Conditioned Media (PWMSCM), and 50 ng/ml rmouse Steel Factor (=Stem Cell Factor, SCF).

### Results

CD26 and CXCR4 Expression

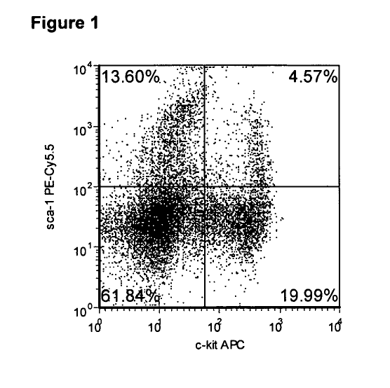

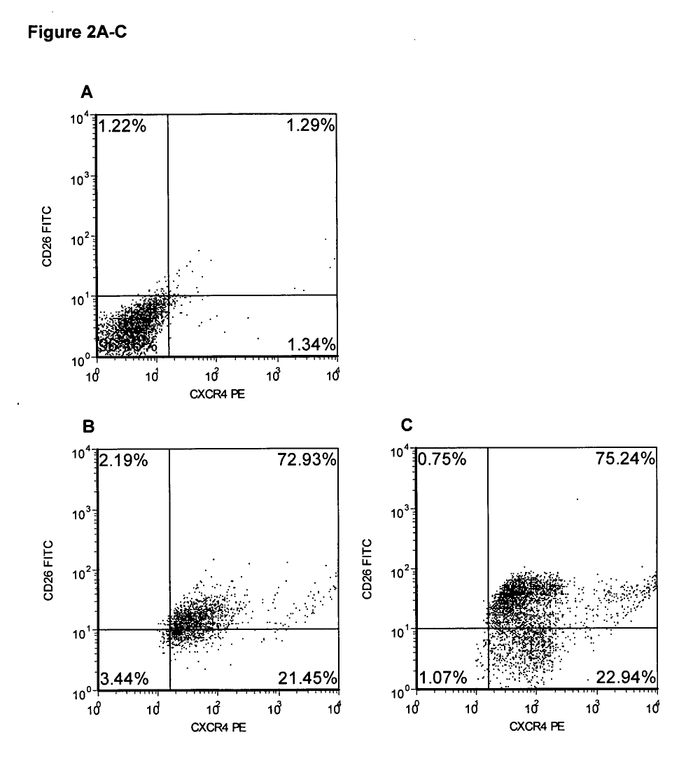

CD26 cell surface expression was measured by multi-variant flow cytometry using fluorochrome-conjugated monoclonal antibodies to mouse CD26, CXCR4, Sca-1, and c-kit. Simultaneous analysis of Sca-1 and c-kit of lineage depleted mBM cells allows for the analysis of Sca-1⁺c-kit⁺lin⁻ cells (FIG. 1, upper-right quadrant) and Sca-1⁺c-kit⁻lin⁻ cells (FIG. 1, upper-left quadrant). CD26 is expressed on approximately 73% of Sca-1⁺c-kit⁺lin⁻ cells (FIG. 2B). Simultaneous examination of CXCR4 expression in these cells reveals that the majority of CD26+ and CD26− cells express CXCR4 (FIG. 2B). Similarly, 75% of Sca-1⁺c-kit⁻lin⁻ cells cells are CD26+ (FIG. 2C) and of those the majority are CXCR4+ (FIG. 2C). In addition, it was noted that Sca-1⁺c-kit⁻lin⁻ cells have distinct CD26+ and CD26− populations, where Sca-1⁺c-kit⁺lin⁻ cells have one population of cells with respect to CD26 expression of which 73% fall in the CD26+ positive category as compared to the isotype control (FIG. 2A).

CD26 Peptidase Activity

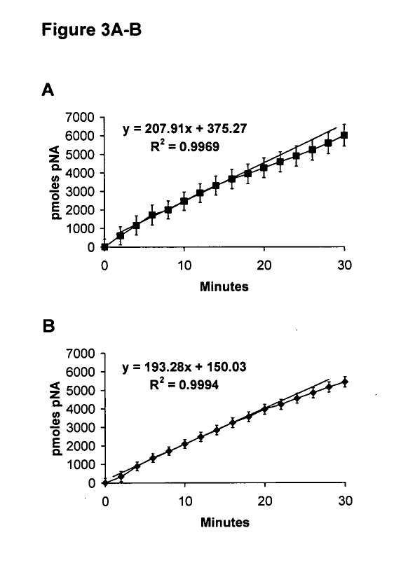

Having shown that a subpopulation of Sca-1⁺c-kit⁺lin⁻ cells and Sca-1⁺c-kit⁻lin⁻ mBM cells existed in which CD26 was expressed, next it was shown that this population of cells had CD26 peptidase activity. Using the chromogenic substrate Gly-Pro-p-nitoanilide (Gly-Pro-pNA), the production of pNA produced by CD26 cleavage was monitored by measuring absorbance at 405 nm. The results of this assay were plotted as pmoles pNA produced versus minutes (FIGS. 3A&B) and the slope was calculated at the linear portion of the enzymatic curve giving a measure of peptidase activity expressed as U/1000 cells where 1 U=1 pmole pNA/min. Sca-1⁺c-kit⁺lin⁻ mBM cells have CD26 peptidase activity and it was measured to be 207.97 U/1000 cells (n=8, FIG. 3A). This is approximately the same activity recorded for Sca-1+c-kit−lin− mBM cells (193.28 U/1000 cells, n=8, FIG. 3B). This data provides evidence that CD26 regulates cellular response to CXCL12 in both Sca-1⁺c-kit⁺lin⁻ and Sca-1⁺c-kit⁻lin⁻ mBM cells.

Migration of mBM HSC/HPC

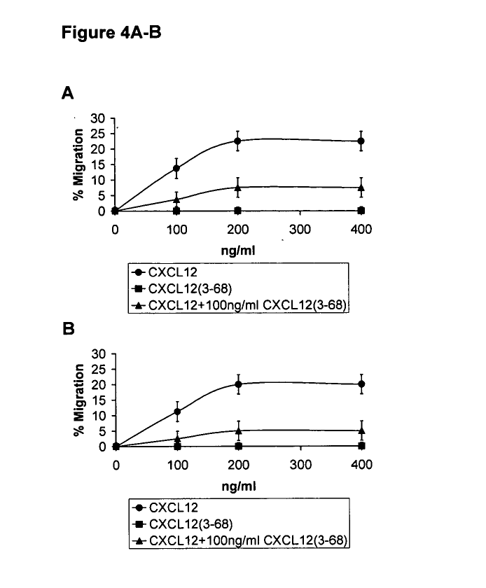

Chemotaxis assays were performed in order to test the functional role of CD26 in HSC/HPC cell migration from normal mBM. Normal Sca-1⁺c-kit⁺lin⁻ cell migration is increased in response to increasing concentration of CXCL12, after incubation at 37° C. for four hours (n=8, FIG. 4A.) The N-terminal truncated CXCL12(3-68), produced by treatment with DPPIV lacks the ability to induce the migration of Sca-1⁺c-kit⁺lin⁻ cells (n=8, FIG. 4A). In addition, 15 minute pretreatment with 100 ng/ml of truncated CXCL12 (3-68) inhibits the normal migratory response at 100 ng/ml of CXCL12 (n=8, p=0.04, FIG. 4A) after four hours from 13.75±4.08% to 3.75±2.88%, representing a 66% loss in percent migration.

Similar results are seen when examining the migration of normal Sca-1⁺c-kit⁻lin⁻ cells. CXCL12 induces a dose dependent chemotaxis, and CXCL12(3-68) lacks the ability to migrate Sca-1⁺c-kit⁻lin⁻ cells (n=8, FIG. 4B). Pretreatment with 100 ng/ml of truncated CXCL12 (3-68) inhibits the normal migratory response at 100 ng/ml of CXCL12 (n=8, p=0.02, FIG. 4B) after four hours from 11.25±3.2% to 2.50±2.39%, representing a 75% loss in percent migration.

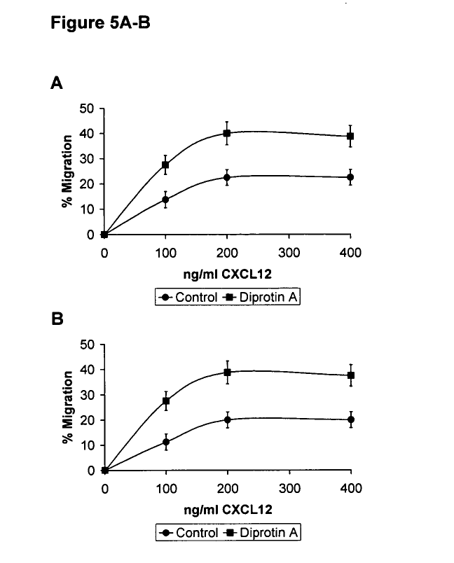

Treatment with 5 mM Diprotin A (Ile-Pro-Ile) was observed to enhance the migratory response of Sca-1⁺c-kit⁺lin⁻ mBM cells to CXCL12 (n=8, p=0.03, FIG. 5A). The enhancement with Diprotin A treatment is equivalent to a 1.7-fold increase in total cell migration in response to 200 and 400 ng/ml CXCL12. When the concentration of CXCL12 is lowered to 100 ng/ml, the enhancement in migration with Diprotin A treatment is two-fold. Treatment with Diprotin A also enhanced the migratory response of Sca-1⁺c-kit⁻lin⁻ mBM cells to CXCL12 (n=8, p=0.02, FIG. 5B). The enhancement with Diprotin A treatment is equivalent to a two-fold increase in total cell migration in response to 200 and 400 ng/ml CXCL12 and 2.5-fold at 100 ng/ml.

Mobilization of HPC

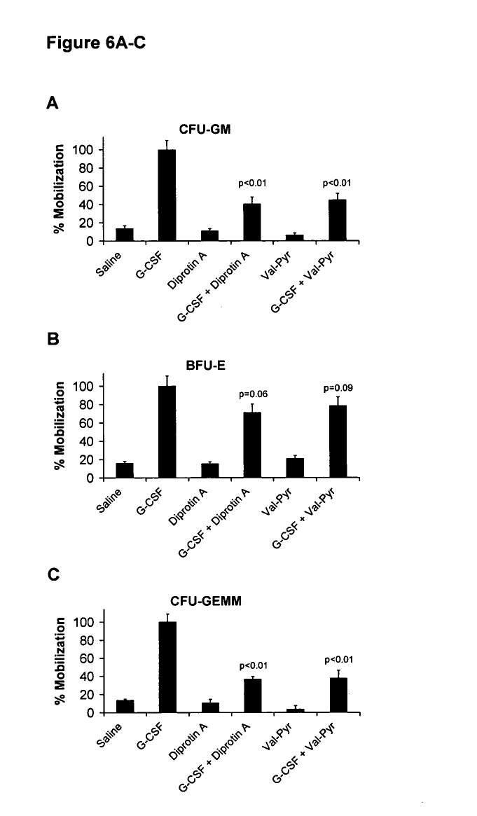

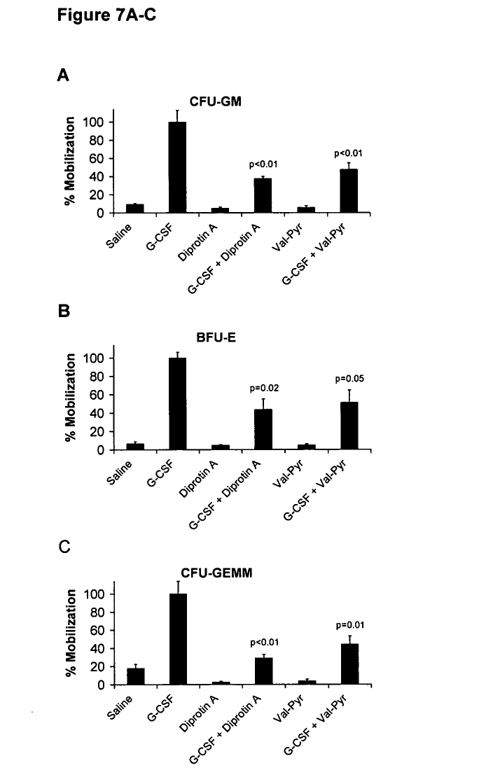

G-CSF induced mobilization of HSC/HPC in C57BL/6 mice was achieved by treating mice with 2.5 μg (micrograms) G-CSF/mouse 2×/day (s.c.) C57BL/6 mice were observed to be relatively poor responders to G-CSF, mobilizing 2348±249 CFU-GM/ml, 1027±107 BFU-E/ml, and 442±35 CFU-GEMM/ml. Data are plotted as a % mobilization, where G-CSF is equal to 100% for each progenitor subtype (FIG. 6A-C). Treatment with either 5 μmol Diprotin A/mouse 2×/day s.c. alone or 5μmol Val-Pyr/mouse 2×/day s.c. alone were observed to have little or no effect on the mobilization of progenitors (FIG. 6A-C). However, Dipotin A treatment during G-CSF mobilization resulted in a 59% reduction in CFU-GM (p<0.01, FIG. 6A), 29% reduction in BFU-E (p=0.06, FIG. 6B)), and 63% reduction in CFU-GEMM (p=0.01, FIG. 6C). Treatment with scrambled peptides (Ile-Ile-Pro and Pro-Ile-Ile) had no effect on mobilization (data not shown). Val-Pyr treatment during G-CSF mobilization resulted in a 55% reduction in CFU-GM (p<0.01, FIG. 6A), 22% reduction in BFU-E (p=0.09, FIG. 6B), and 62% reduction in CFU-GEMM (p<0.01, FIG. 6C) compared to G-CSF alone. G-CSF induced mobilization of HSC/HPC in DBA/2 mice was again achieved by treating mice with 2.5 μg G-CSF/mouse 2×/day s.c. DBA/2 mice were observed to be relatively good responders to G-CSF, mobilizing 8145±1038 CFU-GM/ml, 2219±141 BFU-E/ml, and 1186±163 CFU-GEMM/ml. Data are again plotted as a % mobilization, where G-CSF is equal to 100% for each progenitor subtype. Diprotin A alone or Val-Pyr alone was observed to have little or no effect on the mobilization of progenitors (FIG. 7A-C). However, Dipotin A treatment during G-CSF mobilization resulted in a 62% reduction in CFU-GM (p<0.01, FIG. 7A), 56% reduction in BFU-E (p=0.02, FIG. 7B)), and 71% reduction in CFU-GEMM (p<0.01, FIG. 7C). Val-Pyr treatment during G-CSF mobilization resulted in a 52% reduction in CFU-GM (p<0.01, FIG. 7A), 49% reduction in BFU-E (p=0.05, FIG. 7B), and 56% reduction in CFU-GEMM (p<0.01, FIG. 7C) compared to G-CSF alone.

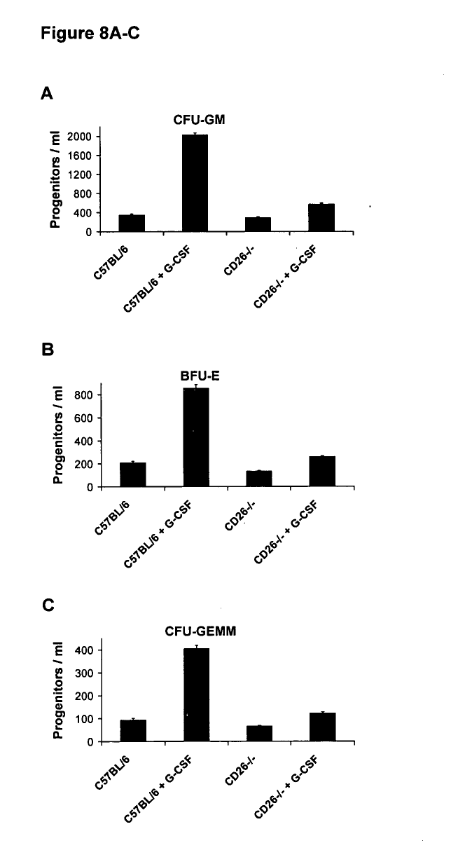

In order to test whether CD26 was an essential component of normal G-CSF induced mobilization of HSC/HPC, in vivo mouse studies were again utilized. Comparison of G-CSF induced mobilization of HPC in WT C57BL/6 mice and CD26^{-/-} mice, also on a C57Bl/6 background, is the best way to accurately assess the importance of CD26 in G-CSF induced mobilization. Assessment of progenitors in the peripheral blood of untreated WT mice and untreated CD26^{-/-} mice revealed that there was no statistical difference in the number of CFU-GM, BFU-E, or CFU-GEMM in the peripheral blood of WT vs. CD26^{-/-} mice (FIGS. 8A-C.)

G-CSF treatment of WT mice resulted in the mobilization of progenitors as expected. G-CSF treatment of CD26^{-/-} mice resulted in low, but significant, mobilization of progenitor cells. The number of progenitors detected in CD26^{-/-} mouse peripheral blood, following G-CSF treatment, were, in general, equivalent to the corresponding number of CFU-GM, BFU-E, and CFU-GEMM detected in the peripheral blood of untreated WT mice. The loss of normal G-CSF induced mobilization of HPC in CD26^{-/-} provides evidence that CD26 is essential for normal G-CSF-induced progenitor cell mobilization. It is believed that CD26^{-/-} mice are the only mice deficient in any one peptidase or gene, other than G-CSFR mice, (Liu F, et al. Blood. 2000;95:3025-3031) that exhibit a deficiency in normal G-CSF induced mobilization of HPC.

### Discussion

CXCL12 chemoattracts HSC/HPC (Aiuti A, et al. J Exp Med. 1997;185:111-120; Kim C H, et al. Blood. 1998;91:100-110; Broxmeyer H E. Int J Hematol. 2001;74:9-17) and is an important component of the mobilization of HSC/HPC from the bone marrow (Broxmeyer H E. Int J Hematol. 2001;74:9-17). CD26 has the ability to cleave CXCL12 after the proline at position two (Lambeir A M, et al. J Biol Chem. 2001;276:29839-29845). The instant inventors recently presented evidence that CD26 is expressed by a subpopulation of normal CD34+ hematopoietic cells isolated from cord blood and that these cells posses CD26 peptidase activity (Christopherson K W, 2nd, et al. J Immunol. 2002;169:7000-7008). More importantly, the functional in vitro studies performed indicate that the process of CXCL12 cleavage by CD26 on a subpopulation of CD34+ cells represents a novel regulatory mechanism for the entire HSC/HPC population with respect to the migration, homing, and mobilization of these cells (Christopherson K W, 2nd, et al. J Immunol. 2002;169:7000-7008).

Since CD26 expression had never been examined in normal bone marrow cells from any source, the expression of CD26 on normal Sca-1⁺c-kit⁺lin⁻ hematopoietic cells isolated from mouse BM was examined. CD26 was determined to be expressed on a significant portion (73%)of Sca-1⁺c-kit⁺lin⁻ cells. Simultaneous examination of CXCR4 expression in these cells revealed that the majority of CD26+ and CD26− cells express CXCR4. Similarly, a significant portion of Sca-1⁺c-kit⁻lin⁻ (75%) cells are CD26+, of which the majority are CXCR4+. These data taken together suggest that the CD26+ subpopulation of either Sca-1⁺c-kit⁺lin⁻ or Sca-1⁺c-kit⁻lin⁻ cells from mBM has the ability to regulate cellular response to CXCL12, and that regulation has in vivo significance, since almost all of the cells expressing CD26 are also CXCR4 positive.

Having shown that a subpopulation of Sca-1⁺c-kit⁺lin⁻ and Sca-1⁺c-kit⁻lin⁻ cells exists that express CD26, the CD26 peptidase activity of these populations of cells was tested using the chromogenic substrate Gly-Pro-p-nitoanilide (Gly-Pro-pNA). Based on the production of pNA by CD26 peptidase cleavage, it was shown that Sca-1⁺c-kit⁺lin⁻ cells isolated from mBM possess CD26 peptidase activity equivalent to 207.97 U/1000 cells (1 U=1 pmole pNA/min). This is approximately the same as the 193.28 U/1000 cells peptidase activity recorded for Sca-1+c-kit−lin− mBM cells. These data establish that not only do Sca-1⁺c-kit⁺lin⁻ and Sca-1⁺c-kit⁻lin⁻ cells express the extracellular peptidase CD26 in an active form but that the activity may have the ability to significantly negatively regulate CXCL12 by N-terminal truncation.

In vitro chemotaxis assays were performed using sorted mouse Sca-1⁺c-kit⁺lin⁻ and Sca-1⁺c-kit⁻lin⁻ cells isolated from mBM in order to test the functional role of CD26. Comparison of Sca-1⁺c-kit⁺lin⁻ cell migration induced by the normal CXCL12 to the truncated CXCL12 (3-68), produced by DPPIV treatment, showed an inability of CXCL12(3-68) to induce chemotaxis. In addition, CXCL12(3-68) acts as an antagonist, resulting in the reduction of Sca-1⁺c-kit⁺lin⁻ cell migratory response to normal CXCL12. Sca-1⁺c-kit⁻lin⁻ cells also did not undergo chemotaxis in response to the truncated CXCL12(3-68), and showed a reduction in CXCL12 stimulated chemotaxis after treatment with CXCL12(3-68). Similar studies using pre-treatment of cells with normal CXCL12 have shown that the CXCR4 receptor can be desensitized, reducing subsequent treatments with CXCL12 (Kim C H, et al. Blood. 1998;91:100-110). The data presented here suggest that the N-terminal truncated form of CXCL12 has no chemotactic activity toward normal Sca-1⁺c-kit⁺lin⁻ or Sca-1⁺c-kit⁻lin⁻ mBM cells but still has the ability to bind the CXCR4 receptor and block migration of cells induced by normal CXCL12.

Treatment of Sca-1⁺c-kit⁺lin⁻ mBM cells with the CD26 inhibitor, Diprotin A, enhanced the migratory response of these cells. Treatment of Sca-1⁺c-kit⁻lin⁻ cells with Diprotin A also enhanced the migratory response of these cells. These data corroborate observations previously made about the enhancement of CXCL12 migration in CD34+ cord blood cells (Christopherson K W, 2nd, et al. J Immunol. 2002;169:7000-7008). These in vitro observations also suggest that treatment with the CD26 inhibitor is blocking the endogenous CD26 peptidase activity expressed on the surface of a subpopulation of these cells, resulting in a change in functional activity. Clearly, CD26 has the ability to negatively regulate CXCL12 signaling through the CXCR4 receptor in normal mouse HSC/HPC by cleaving local pools of CXCL12. This indicates that CD26 expressed on the surface of a subpopulation of HSC/HPC collectively has the ability to self-regulate its own cellular response to CXCL12 as well as the cellular response of surrounding HSC/HPC. CXCL12 is an important chemokine involved in the homing/mobilization of HSC/HPC to/from the bone marrow (Broxmeyer H, Smith F. Cord Blood Stem Cell Transplantation. In: Thomas E D, ed. Hematopoietic cell transplantation (ed 2nd) Oxford; Malden, Mass., USA: Blackwell Science; 1999:431-443; Peled A, et al. Science. 1999;283:845-848; Petit I, et al. Nat Immunol. 2002;3:687-694). It has been proposed by others that direct degradation of CXCL12 by proteolytic enzymes, including neutrophil elastase and cathepsin G, may play a role in HSC/HPC mobilization (Petit I, et al. Nat Immunol. 2002;3:687-694). To test for potential in vivo relevance of CD26 cleavage of CXCL12 in the context of G-CSF induced mobilization, mice were co-treated with CD26 inhibitors during G-CSF induced mobilization. As previously noted by others, (Roberts A W, et al. Blood. 1997;89:2736-2744; Hasegawa M, et al. Blood. 2000;95:1872-1874) G-CSF induced mobilization of HPC in the absence of CD26 inhibitors in C57BL/6 mice was observed to be relatively poor compared to G-CSF mobilization in DBA/2 mice.

In order to make comparisons of inhibition of mobilization between mouse strains, data was expressed as % mobilization, with G-CSF alone equal to 100% for each strain. Treatment with either CD26 inhibitor alone (Diprotin A or Val-Pyr) was observed to have little or no effect on the mobilization of progenitors in either C57BL/6 or DBA/2 mice. However, co-treatment with Diprotin A during G-CSF mobilization resulted in a significant reduction in CFU-GM, BFU-E, and CFU-GEMM in the peripheral blood. Treatment with a second CD26 inhibitor (Val-Pyr), at equivalent molar concentrations of Diprotin A used, during G-CSF induced mobilization resulted in a significant reduction of CFU-GM, BFU-E, and CFU-GEMM in the peripheral blood almost equivalent to that seen with Diprotin A co-treatment. The use of a second CD26 inhibitor during G-CSF induced mobilization provides further support for the hypothesis that the reduction in HPC observed in the periphery as compared to the G-CSF regiment alone is the result of specifically inhibiting CD26 activity. This reduction in the number of progenitor cells mobilized suggests that a mechanism of action of G-CSF mobilization involves CD26. The % reduction in HPC mobilized during CD26 inhibitor co-treatment was greater in DBA/2 mice than C57BL/6 mice, possibly reflecting an increased role of CD26 in G-CSF mobilization in DBA/2 mice. An increased role of CD26 in the response of DBA/2 mice to G-CSF treatment compared to C57BL/6 mice has not been previously suggested. However, linkage analysis studies have indirectly suggested that the difference in HPC mobilization observed between DBA/2 and C57BL/6 mice is due to genes located in a region on mouse Chromosome 2 between genetic markers D2Mit83 (Hasegawa M, et al. Blood. 2000;95:1872-1874) at 16.0 cM (MGI:94919. Mouse Genome Database (MGD): Mouse Genome Informatics, The Jackson Laboratory, Bar Harbor, Me.; 2002) and D2Mit229 (Hasegawa M, et al. Blood. 2000;95:1872-1874) at 99.0 cM (MGI:94919. Mouse Genome Database (MGD): Mouse Genome Informatics, The Jackson Laboratory, Bar Harbor, Me.; 2002). Interestingly, the mouse CD26 gene, Dpp4, is located on mouse Chromosome 2 at 35.0 cM, (MGI:94919. Mouse Genome Database (MGD): Mouse Genome Informatics, The Jackson Laboratory, Bar Harbor, Me.; 2002) which falls within this region suggested to be important by linkage analysis (Hasegawa M, et al. Blood. 2000;95:1872-1874). CD26 cleavage of CXCL12 results in the formation of a N-terminal truncated CXCL12(3-68). This cleaved form of CXCL12 lacks migratory ability and inhibits the migratory ability of normal CXCL12. In this way, it is possible for CD26 expressed on a sub-population of cells to inhibit the migration of all HSC/HPC within a local pool of cells. The process of CXCL12 cleavage by CD26 may represent a novel regulatory mechanism in hematopoietic stem cells for the migration, homing, and mobilization of these cells. Presented here is in vivo evidence that implicates CD26 in G-CSF induced mobilization of HPC.

### EXAMPLE 2

### Methods of Improving Stem Cell Homing and Engraftment Efficiency

### Materials and Methods

Preparation of Mouse Bone Marrow Cells

Mouse bone marrow (BM) cells were flushed from femurs of 6-8 week old mice and low density (LD) cells were isolated by density centrifugation using Lympholyte M (Stem Cell Technologies, Vancouver, BC). Normal C57BL/6 mice were purchased from Harlan (Indianapolis, Ind.) and congenic BoyJ mice were purchased from Jackson Labs (Bar Harbor, Me.). Mice deficient in the expression of CD26/DPPIV (CD26^{-/-} mice) (on a C57Bl/6 background) were obtained from Dr. Nicolai Wagtmann (Novo Nordisk, Denmark) with approval of Dr. D. Marguet (Marguet D, et al. Proc Natl Acad Sci USA. 2000;97:6874-6879).

CD26 Inhibitors

Two specific inhibitors of CD26 peptidase activity were utilized in the following experiments. Diprotin A, a three amino acid peptide (Ile-Pro-Ile), was purchased from Peptides International (Louisville, Ky.). Valine-Pyrrolidide (Val-Pyr) was obtained as a gift from Dr. Nicolai Wagtmann (Novo Nordisk, Denmark).

CXCR4 Inhibitor.

The CXCR4 chemokine receptor non-peptide antagonist, AMD3100 (Rosenkilde M. M., et al., J Biol Chem 2004; 279, 3033) was provided as a gift from AnorMED (Langley, BC, Canada).

CD26/DPPIV Peptidase Activity

CD26 peptidase activity of sorted cells was measured in 96 well microplates using the chromogenic substrate Gly-Pro-p-nitoanilide (Gly-Pro-pNA) (Sigma, St Louis, Mo.) as previously reported (Christopherson K W, 2nd, et al. J Immunol. 2002;169:7000-7008; Kojima K, et al. J Chromatogr. 1980;189:233-240; Nagatsu T, et al. Anal Biochem. 1976;74:466-476.) Peptidase activity is expressed as pmoles/min (U) per 1000 cells. Proteolytic activity was determined by measurement of the amount of p-nitroanilide (pNA) formed in the supernatant at 405 nm. One thousand cells per well in the 96-well flat-bottomed plate were incubated at 37° C. with 4 mM Gly-Pro-pNA in 100 μL PBS buffer (pH 7.4) containing 10 mg/ml BSA. Absorbance was measured at 405 nm on a microplate spectrofluorometer (SpectraMax 190, Molecular Devices, Sunnyvale, Calif.) every two minutes and pmoles of pNA formed were calculated by comparison to a pNA standard curve. The results were plotted as pmoles pNA versus minutes and the slope was calculated at the linear portion of the curve giving a measure of DPPIV activity expressed as pmoles/min (U) per 1000 cells. Tests were run using three separate samples (n=3 for each sample); cell-free blanks, substrate-free blanks were run in parallel.

Migration of HSC

Chemotaxis assays were performed using 96-well chemotaxis chambers (NeuroProbe, Gaithersburg, Md.) in accordance with manufacturer's instructions as described previously with minor variations (Christopherson K W, 2nd, et al. J Immunol. 2002;169:7000-7008; Christopherson K W, 2nd, et al. Blood. 2001;98:3562-3568; Christopherson K, 2nd, et al. Immunol Lett. 1999;69:269-273). Briefly 300 μl of RPMI supplemented with 10% FBS and either 0, 100, 200, and 400 ng/ml of mouse CXCL12 chemokine was added to the lower chamber. Ten-thousand sorted cells in 50 μL of media were added to the upper side of the membrane (5.7 mm diameter, 5 μm pore size, polycarbonate membrane.)

Total cell number in the lower well was obtained by counting using hemocytometer after 4 hours of incubation at 37° C., 5% CO₂. Percent migration was calculated by dividing the number of the cells in the lower well by the total cell input multiplied by 100 and subtracting random migration (always less than 5%) to the lower chamber without chemokine presence. Three samples were analyzed separately in triplicate, and then the data averaged for statistical analysis.

Inhibition of endogenous CD26/DPPIV activity was accomplished by pretreatment of cells with 5 mM Diprotin A (Peptides International, Louisville, Ky.) for 15 minutes at 37° C. Diprotin A was allowed to remain in the chemotaxis chamber during the assay. Chemotaxis assays were performed with and without Diprotin A in conjunction with a CXCL12 dose response between 0 and 400 ng/ml.

Short-Term Homing

Control C57BL/6 (CD45.2⁺), CD26 inhibitor treated C57BL/6 (CD45.2⁺), or CD26^{-/-} (CD45.2⁺) sorted Sca-1⁺lin⁻ or LD donor BM cells were transplanted by tail-vein injection into lethally irradiated (1100cGy split dose) congenic BoyJ (CD45.1⁺) female recipient mice(Haneline L S, et al. Blood. 1999;94:1-8.) Contribution of Sca+lin⁻ donor cells in the context of Sca-1⁺lin⁻ recipient cells was calculated by flow cytometric analysis of cells found in the recipient mouse BM by measuring CD45.1⁺ and CD45.2⁺ on Sca-1⁺lin⁻ LD cells isolated from the recipient mice 24 hours post transplant. No less than five mice from each recipient group were analyzed, each one having received a transplant of 20×10⁵ pooled LD donor BM cells. Comparisons between treatment groups were made using a two-tailed Student's t-test and data was plotted as mean (M)±standard error of the mean (SEM).

Long-Term Engraftment

Control C57BL/6 (CD45.2⁺) or CD26 inhibitor treated C57BL/6 (CD45.2⁺) LD donor BM cells were transplanted by tail-vein injection into lethally irradiated (1100cGy split dose) congenic BoyJ (CD45.1⁺) female recipient mice (Haneline L S, et al. Blood. 1999;94:1-8). Contribution of donor cells was obtained by measuring the number of donor and residual recipient lymphocytes in the peripheral blood. The % donor contribution was calculated by flow cytometric analysis of peripheral blood lymphocytes found in the recipient mouse peripheral blood by measuring CD45.1⁺ and CD45.2⁺ on cells isolated from the recipient mice 1-6 months post transplant. No less than five mice from each recipient group were analyzed, each one having received a transplant of 5×10⁵ pooled LD donor BM cells. Comparisons between treatment groups were made using a two-tailed Student's t-test and data was plotted as mean (M)±standard error of the mean (SEM).

Secondary Transplantation.

Secondary transplants are performed to assess long-term engraftment capabilities of cells able to exhibiting self-renewal capacity. Donor LDBM cells (5×10⁵) from representative mice from each test group were transplanted into lethally irradiated BoyJ mice (expressing CD45.1) Quantitative contribution to chimerism was obtained 2-6 months post-transplantation in the same manner described for long-term engraftment assays.

Competitive Repopulation Assay

Eight week old female recipient mice were lethally irradiated (1100cGy split dose) prior to transplantation as previously described (Haneline L S, et al. Blood. 1999;94:1-8). Limiting dilutions of donor LD BM cells (5×10⁵, 2.5×10⁵, 1.25×10⁵, and 0.625×10⁵ cells) from control C57Bl/6 mice, CD26-inhibitor treated LD BM cells, or untreated LD BM cells from CD26^{-/-} mice (all expressing CD45.2) are co-transplanted with a constant number (5×10⁵) of pooled competitive BM cells from congenic BoyJ mice (expressing CD45.1), into BoyJ irradiated mice. To determine quantitatively the relative cell contribution to chimerism of each test cell population tail-vein blood samples were obtained post-transplantation each month for three months and CD45.1 and CD45.2 was measured by flow-cytometry. Cells were divided into CD26 inhibitor (either Diprotin A or Val-Pyr) treated or untreated groups obtained from normal C57BL/6 mice and untreated cells obtained from CD26^{-/-} mice. Comparisons between treatment groups were made using a two-tailed Student's t-test and data was plotted as mean (M)±standard error of the mean (SEM)

Transplant Recipient Survival Curve

Limiting dilution survival curve experiments were performed to assess changes in survival rates of recipient mice during transplantation. This is especially important in the context of transplantation of small numbers, limiting dilutions of donor cells. Mice were transplanted with a limiting dilution (2×10⁵, 1×10⁵, 5×10⁴, 2.5×10⁴ cells per recipient mouse) of donor cells. Survival was monitored daily and data were plotted as % survival for each given cell dose.

### Results

HSC Migration

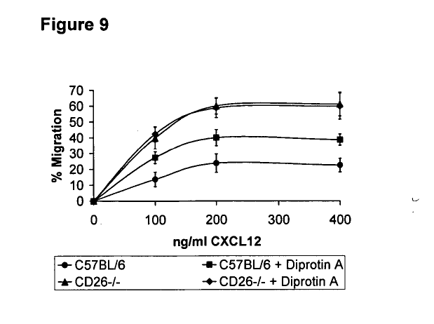

HSC isolated from mouse BM were defined as cells contained within a larger Sca-1⁺lin⁻ population. We previously established that Diprotin A (Ile-Pro-Ile) is a specific inhibitor of CD26 using chemotaxis assays. We show here that Diprotin A treated Sca-1⁺lin⁻ BM cells from C57BL/6 mice exhibited a two-fold increase in CXCL12-induced migratory response (FIG. 9). Similarly, CD26 deficient (CD26^{-/-}) Sca-1⁺lin⁻ BM cells possessed up to a three-fold greater migratory response, as compared to control C57BL/6 Sca-1⁺lin⁻ BM cells (FIG. 9). Diprotin A treatment of CD26^{-/-} cells had no further enhancing effect compared to untreated CD26^{-/-} cells (FIG. 9). Thus, the in vitro migratory response of Sca-1⁺lin⁻ HSC cells to CXCL12 was enhanced by specific inhibition and even more significantly by a complete absence of CD26 peptidase activity.

Short-term Homing

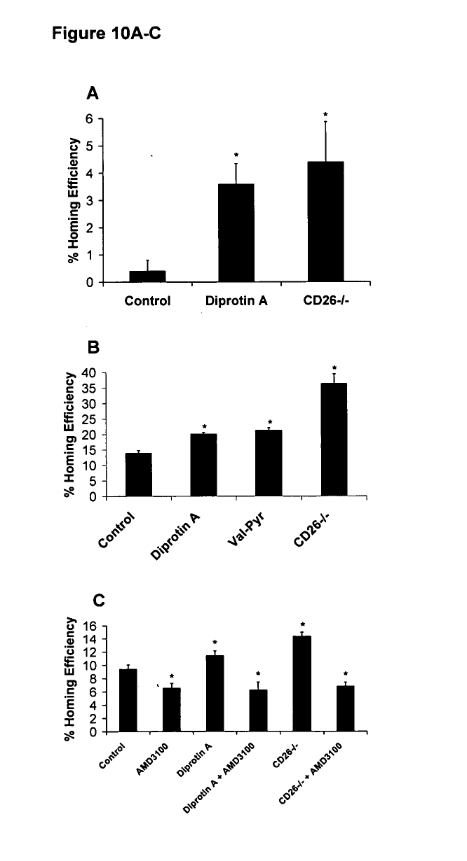

Short-term homing experiments were undertaken using a modified congenic mouse model in which C57Bl/6 (CD45.2⁺) and BoyJ (CD45.1⁺) cells can be disseminated to assess recruitment of HSC to the bone marrow following transplantation. Treatment of 1×10⁴ sorted Sca-1⁺lin⁻ BM C57Bl/6 donor cells with the CD26 inhibitor (Diprotin A) for 15 minutes prior to transplant resulted in a 9-fold increase in homing efficiency of these cells in Boy/J recipient mice as compared with untreated cells (FIG. 10A). Similarly, transplantation of sorted Sca-1⁺lin⁻ BM cells from CD26^{-/-} mice resulted in an 11-fold increase in homing efficiency to recipient BM (FIG. 10A). These data suggest that inhibition, or loss of CD26 activity results in a significant increase in homing of sorted Sca-1⁺lin⁻ HSC cells in vivo. Treatment of 20×10⁶ low density bone marrow (LDBM) donor cells with CD26 inhibitors prior to transplant, resulted in a 1.5-fold increase in homing efficiency of C57BL/6 Sca-1⁺lin⁻ cells within the LDBM donor cells into BoyJ recipient mouse BM 24 hours post transplant (FIG. 10B). Transplantation of CD26^{-/-} cells provided a 2.6-fold increase in homing efficiency of Sca-1⁺lin⁻ cells within the LDBM donor cells, as compared to control wild-type (WT) cells (FIG. 10B). Thus, inhibition or loss of CD26 activity in the total LDBM donor unit (which contains large numbers of fully differentiated cells as well as HPC) results in an increase in in vivo homing of Sca-1⁺lin⁻ HSC cells within that LDBM donor unit. Transplantation of LDBM cells was performed because this represents a more accurate depiction of clinical transplantation protocols than transplantation of sorted Sca-1⁺lin⁻ HSC. The differences in homing efficiency between sorted Sca-1⁺lin⁻ cells and LDBM cells may be partially explained by larger numbers of Sca-1⁺lin⁻ donor cells (3×10⁴) contained within the 20×10⁶ cell LDBM donor unit. Alternatively, the differences may be attributable to accessory cells contained within the LDBM unit that are not present in sorted Sca-1⁺lin⁻ cells.

CD26 Peptidase Activity

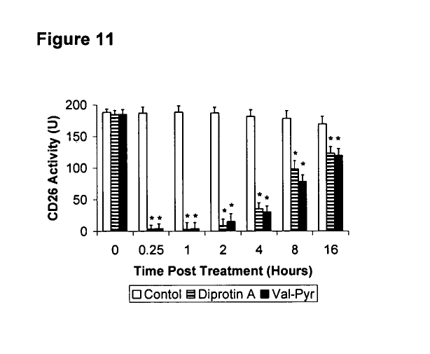

Treatment of 10×10⁶ LDBM donor cells with the CXCR4 antagonist, AMD3100, for 15 minutes prior to transplantation reversed the increase in homing efficiency of Sca-1⁺lin⁻ cells generated by inhibition or loss of CD26 (FIG. 10C). AMD3100 treatment alone also resulted in a reduction in homing efficiency, as compared to control LDBM cells (FIG. 10C). Treatment with the CXCR4 antagonist, AMD3100 and in vitro CD26^{-/-} HSC/HPC migration data, combined with our previous studies involving CD26 inhibitors, suggest that CXCL12 is a logical downstream target of the observed enhancement of transplant efficiency. This is reasonable since CXCL12 is believed to play an important role in migration, mobilization, homing and engraftment of HSC. CXCL12, has also been suggested to play an important role in the mechanism responsible for holding HSC/HPC in the bone marrow and providing signals for enhancing cell survival, an additional component of HSC engraftment. Although CD26 peptidase activity is rapidly lost with treatment of either Diprotin A or Val-Pyr, recovery of CD26 activity was however noted to begin within four hours post treatment (FIG. 11). The rapid recovery of CD26 activity could explain the short-comings of inhibitor treated donor cells as compared to CD26^{-/-} donor cells observed during short-term homing experiments (FIG. 10A & 10B).

Long-term Engraftment and Mouse Survival

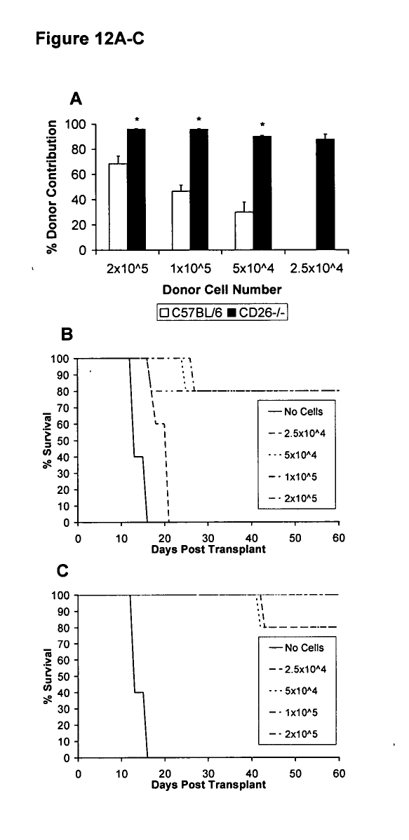

To assess transplant efficiency it is necessary to consider the long-term engraftment capacity of donor HSC. Transplants were performed with 5×10⁵ LDBM C57Bl/6 (CD45.2⁺) or CD26^{-/-} (CD45.2⁺) cells and congenic BoyJ (CD45.1⁺) recipients. In these experiments CD26^{-/-} donor cells made a significantly greater contribution to peripheral blood leukocytes as determined six months after transplant (FIG. 12A). This was revealed especially at limiting dilutions (FIG. 12A) and correlated with changes in mouse survival (FIGS. 12B&C). At day 60, 0% survival was observed in mice transplanted with 2.5×10⁴ control C57BL/6 cells (FIG. 12B), whereas 80% survival was observed in mice transplanted with an equivalent number of CD26^{-/-} mouse cells (FIG. 12C). In this model, transplantation of 2.5×10⁴ normal LDBM cells was below the lower limit of cells required for the level of repopulation necessary for overall mouse survival. Recipient survival is dependent on both short- and long-term reconstitution of the bone marrow. The absence of surviving mice in this group by day 21 may suggest that a loss of short-term reconstitution may be responsible for the lethality of transplant at this dose of donor cells. At limiting numbers of transplanted donor cells, both long-term engraftment and mouse survival increased when CD26^{-/-} donor cells were transplanted. At non-limiting numbers of donor cells (2×10⁵) an improvement is also observed in engraftment and survival with CD26^{-/-} cells at day 60, suggesting that the observed effect may also target long-term reconstitution.

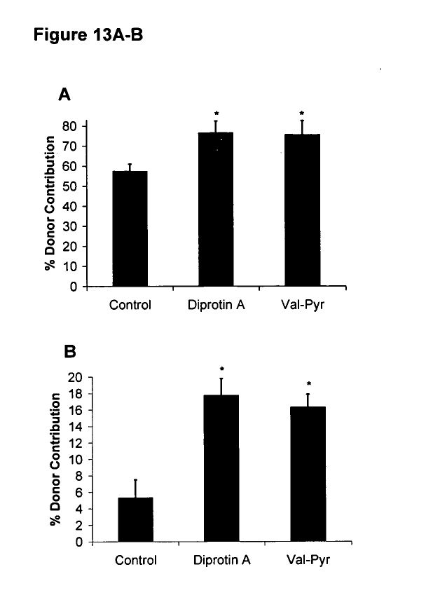

Treatment of C57BL/6 donor cells with either CD26 inhibitor at non-limiting cell doses and in the context of a non-competitive assay resulted in an increase of about one third in donor cell contribution to leukocyte formation in lethally irradiated congenic BoyJ recipient mice relative to untreated cells (FIG. 13A). In secondary transplanted recipient mice, a three-fold increase in donor cell contribution to PB leukocytes was seen using CD26 inhibition (FIG. 13B). The increase in secondary repopulating HSC observed, as compared to repopulating HSC from primary transplant recipients, is indicative of an increase in the homing and engraftment of self-renewing stem cells with CD26 inhibitor treatment.

Competitive Repopulation

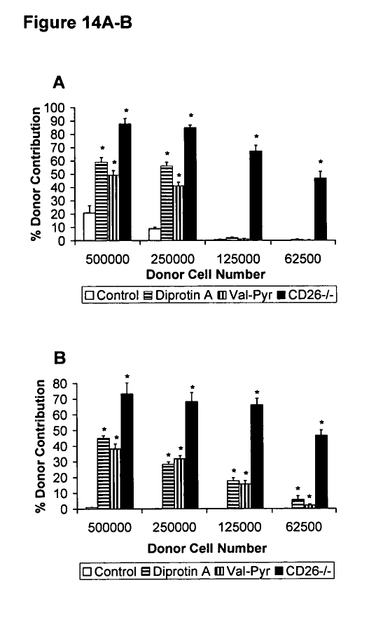

Competitive repopulation assays were conducted to assess long-term engraftment capabilities of experimental donor cell populations in direct comparison with control donor cells. Long-term competitive repopulating HSC assays are believed to provide the most functional assessment of engraftment capabilities of HSC by allowing a direct comparison of the engraftment capacity of HSC from experimental donor cells (CD45.2⁺) relative to a constant number of competitive donor cells (CD45.1⁺) (Haneline L S, et al. Blood. 1999;94:1-8; Harrison D E, et al. Blood. 1980;55:77-81; Szilvassy S J, et al. Proc Natl Acad Sci USA. 1990;87:8736-8740; Bodine D M, et al. Blood. 1996;88:89-97; Yoder M C, et al. Immunity. 1997;7:335-344). At six months post transplant, increased donor contribution to chimerism was observed with CD26 inhibitor (Diprotin A or Val-Pyr) treatment relative to co-transplanted cells (FIG. 14A). At limiting donor cell numbers (1.25×10⁵ and 0.625×10⁵) no significant increases in percent donor contribution was observed with CD26 inhibitor treatment (FIG. 14A). However, CD26^{-/-} donor cells had a significantly enhanced contribution to chimerism at all donor cell numbers measured (FIG. 14A). An even greater increase in donor cell contribution was observed with CD26 inhibitor-treated donor cells and CD26^{-/-} donor cells in secondary transplanted recipient BoyJ mouse PB four months post transplant (FIG. 14B). Inhibitor treatment reflected a strong increase in secondary repopulating stem cells and this was even more striking when CD26^{-/-} donor cells were used in secondary repopulating assays (FIG. 14B).

### Discussion

These results demonstrate that administration of CD26 inhibitors to stem cells in vitro prior to transplantation improves stem cell engraftment efficiency.

Transplantation of hematopoietic stem (HSC) and progenitor cells (HPC) is an inefficient process. There are therefore benefits to increasing the efficiency of transplantation, especially when transplantable cell number is limiting. Through the use of CD26 inhibitors and mice deficient in CD26 (CD26^{-/-}), it is shown herein that reduction or loss of CD26 activity at the level of the donor cell population correlates to an increased efficiency of HSC transplantation. This increase in efficiency manifests itself in the form of increased short-term homing, increased long-term engraftment, increased competitive repopulation, and increased survival rate. Since CD26 is also expressed in recipient tissues, inhibition of CD26 at the level of the recipient is likely to result in increases in the efficiency of transplantation. In light of the foregoing, it can be seen that inhibition of CD26 activity on either the donor cell population or in the transplant recipient is a novel therapeutic tool for the increasing the efficiency of transplantation of hematopoietic stem and progenitor cells, whether they be bone marrow HSC/HPC, mobilized HSC/HPC, or cord blood HSC/HPC.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. A method of enhancing mobilization and engraftment of hematopoietic stem cells (HSC) comprising;
a) isolating HSC;
b) contacting said HSC for a period insufficient for cell division to occur with a CD26 peptidase inhibitor in an amount effective to inhibit CD26 peptidase activity
c) administering HSC so treated to a patient in need thereof.

2. The method of claim 1, wherein the CD26 inhibitor is selected from the group consisting of Diprotin A (Ile-Pro-Ile) and Valine-Pyrrolidide.

3. The method of claim 1, wherein the CD26 inhibitor is administered from about 5 minutes to about 12 hours.

4. The method of claim 1, wherein the CD26 inhibitor is administered from about 15 minutes to about 6 hours.

5. The method of claim 1, wherein the CD26 inhibitor is administered for less than 6 hours.

6. The method of claim 1, wherein the CD26 inhibitor is administered for less than 2 hours.

7. The method of claim 1, wherein the CD26 inhibitor is administered for less than 1 hour.

8. The method of claim 1, wherein the inhibitor is administered in a concentration of no less than about 5 mM.

9. The method of claim 1, wherein the cells are treated at a concentration of about 1×10⁶ donor cells per mL.

10. The method of claim 1 wherein said cells are administered to a patient for a bone marrow transplant, optionally comprising administering a CD26 inhibitor to said patient in an amount effective to inhibit the peptidase activity thereof prior to or during said transplant.

11. The method of claim 10, wherein the inhibitor is administered to said patient in a concentration of about 1 to about 50 μMol/kg total body weight.

12. The method of claim 10, wherein the inhibitor is administered to said patient in a concentration of about 1 to about 30 μMol/kg total body weight.

13. The method of claim 10, wherein the inhibitor is administered to said patient in a concentration of about 1 to about 10 μMol/kg total body weight.

14. An isolated stem cell which has been exposed to a CD26 inhibitor in vitro.
